SEQUENCE LISTING (1 sequence)

SEQ ID NO: 1 - protein, 16 aa, Artificial Sequence
  Description of Artificial Sequence fragment3 1-16 of human adrocorticotropic hormone
SYSMEHFRWGKPVGKK

(19) United States
(12) Patent Application Publication
(10) Pub. No.: US 2004/0043920 A1
(43) Pub. Date: Mar. 4, 2004

(54) Compound for chelating a metal, radiopharmaceutical, manufacturing process therefor, and diagnostic kit

(76) Inventors: Laurent Mauclaire, Paris (FR); Eric Berthommier, Paris (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/432,995

(22) PCT Filed: Nov. 12, 2001
(86) PCT No.: PCT/IB01/02141

(30) Foreign Application Priority Data
Nov. 29, 2000 (FR) 00/15416

(51) Int. Cl. A61K 38/00; C07K 14/00; A61K 31/445; A61K 31/195; A61K 31/277

(52) U.S. Cl. 514/2; 514/562; 530/324; 530/400; 562/556; 564/162

(57) ABSTRACT

The present invention provides a compound for chelating a metal or a metal complex, characterized in that it consists of a bis-dithiocarbamate structure (F) having the formula below:

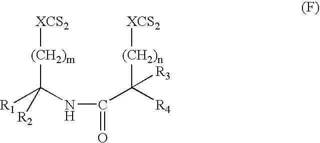

in which n and m are integers such that 5≦m+n≦10,

X is chosen independently from S and NH,

R₁, R₂, R₃ and R₄ are chosen independently from H and an organic function chosen from —COOR₅, NR₅R₆ and —CH₂OR₅ in which R₅ and R₆, when it is present, are chosen independently from a hydrogen; an amino acid; a peptide; a protein; monoclonal antibody; a hormone; and a pharmaceutically acceptable vector.

The present invention also provides a diagnostic kit comprising a chelating compound according to the present invention.

### DESCRIPTION

1. Technical Field of the Invention

The present invention relates to a compound for chelating a metal or a metal complex, to a radiopharmaceutical, to a manufacturing process therefor and to a diagnostic kit.

The chelation compound may be used to manufacture a diagnostic product or a medicinal product.

The metal may be a transition metal chosen, for example, from Tc, Ru, Co, Cu, Pt, Fe, Os, Ir, Re, Cr, Mo, Mn, Ni, Rh, Pd, Nb, Sm and Ta or an isotope thereof.

The metal complex may be, for example, a nitride complex of radioactive transition metals which may be used as radiopharmaceutical products for diagnosis or therapy.

Among the complexes which may be used for diagnosis, mention may be made in particular of technetium 99m complexes.

Radiopharmaceutical products using the ^{99m}Tc radionucleide are very useful in nuclear medicine for diagnosis on account of its physical and chemical characteristics. Technetium complexes which may be used for the present invention are described, for example, by E. DEUTSCH et al. in: Progr. Inorg. Chem. (Australia), vol. 30, pp. 76-106, 1983, and preparation processes are described in J. Baldas et al. in J. Chem.

Soc. Dalton Trans 1981, pp. 1798-1801; in Int. Appl. Radiot. Isot. 36 (1985), pp. 133-139, in international patent application WO 85/03063 and in patent applications EP-A-537 242 and EP-A-0 403 524.

The complexes which may be used for therapy may be, for example, rhenium complexes.

Copper or an isotope thereof is useful for the present invention, for example for labelling antibodies or peptides, for diagnosis and especially for therapy (⁶⁷Co ⁶⁴Cu).

2. Description of the Invention

The compound for chelating a metal or a metal complex of the present invention is characterized in that it consists of a bis-dithiocarbamate structure (F) having the following formula:

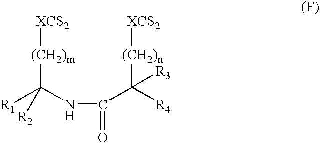

in which n and m are integers such that 5≦m+n≦10,

X is chosen independently from S and NH,

R₁, R₂, R₃ and R₄ are chosen independently from H and an organic function chosen from —COOR₅, NR₅R₆ and —CH₂OR₅ in which R₅ and R₆, when it is present, are chosen independently from a hydrogen; an amino acid; a peptide; a protein; an organic function; a group chosen from alkoxycarbonyl or aryloxycarbonyl (—COOR⁷), carboxyl (—COOH), acyloxy (—O₂R⁷), carbamoyl (—CONR⁷), cyano (—CN), alkylcarbonyl, alkylarylcarbonyl, arylcarbonyl, arylalkylcarbonyl, hydroxyl (—OH), amino (NR⁷), halogen, allyl, alkoxy (—OR⁷), S-alkyl and S-aryl, R⁷ representing a C₁ to C₁₀ alkyl or aryl group; an organic molecule chosen from (i) an optionally substituted alkyl, acyl, aryl or alkyne group, (ii) a saturated or unsaturated, optionally substituted or aromatic carbon-based ring or (iii) a saturated or unsaturated, optionally substituted or aromatic heterocycle, these groups and rings (i), (ii) and (iii) possibly being substituted with substituted phenyl groups, substituted aromatic groups or alkoxycarbonyl or aryloxycarbonyl (—COOR⁸), carboxyl (—COOH), acyloxy (—O₂R⁸), carbamoyl (—CONR⁸), alkylcarbonyl, alkylarylcarbonyl, arylcarbonyl, arylalkylcarbonyl, hydroxyl (—OH), amino (NR⁸), halogen, allyl, alkoxy (—OR⁸), S-alkyl or S-aryl groups, R⁸ representing a C, to C₁₀ alkyl or aryl group; a monoclonal antibody; a hormone; and a pharmaceutically acceptable vector.

n and m are natural integers. Thus, m and n may be, independently, 0, 1, 2, 3, 4, 5, 6, 7, 8, 9 or 10, provided that 5≦m+n≦10. For example, for m=0, n may be 5, 6, 7, 8, 9 or 10, and for m=1, n may be 4, 5, 6, 7, 8 or 9. According to the invention, the functions XCS₂ are in fact separated by a peptide bond and a number of carbon atoms greater than or equal to 7 and less than or equal to 10.

The present invention provides a novel system for complexing a metal or a metal complex which may be linked to any molecule or biomolecule. This system thus has numerous applications, especially for diagnosis and therapy.

According to the invention, the bis-dithiocarbamate compound may consist of a structure whose formula is chosen from formulae (I), (II), (III) and (IV) below:

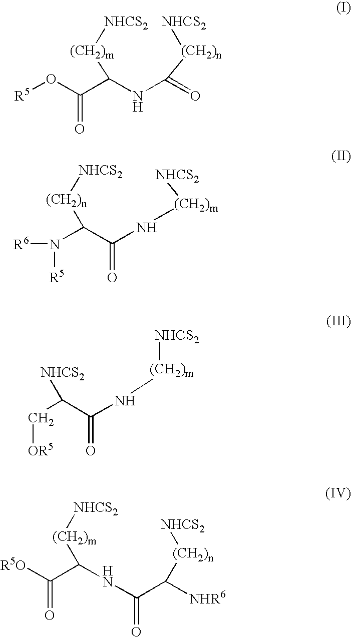

in which n and R⁵, and m and R⁶, when they are present, are as defined above.

According to the invention, R⁵ may be chosen from H, CH₃, a tropane derivative or a compound of formula:

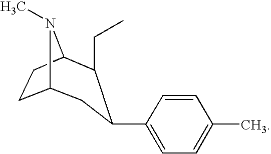

When R⁵ is a tropane derivative, it may be of formula (G) below:

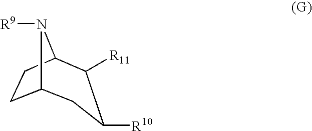

in which one of the radicals R⁹, R¹⁰ and R¹¹ is a compound of formula F, the other radicals being chosen independently from a hydrogen; an organic function; a group chosen from alkoxycarbonyl or aryloxycarbonyl (—COOR⁷), carboxyl (—COOH), acyloxy (—O₂R⁷), carbamoyl (—CONR⁷), cyano (—CN), alkylcarbonyl, alkylarylcarbonyl, arylcarbonyl, arylalkylcarbonyl, hydroxyl (—OH), amino (NR⁷), halogen, allyl, alkoxy (—OR⁷) S-alkyl and S-aryl, R⁷ representing a C₁ to C₁₀ alkyl or aryl group; an organic molecule chosen from (i) an optionally substituted alkyl, acyl, aryl or alkyne group, (ii) a saturated or unsaturated, optionally substituted or aromatic carbon-based ring or (ii) a saturated or unsaturated, optionally substituted or aromatic heterocycle, these groups and rings (i), (ii) and (iii) possibly being substituted with substituted phenyl groups, substituted aromatic groups or alkoxycarbonyl or aryloxycarbonyl (—COOR⁸), carboxyl (—COOH), acyloxy (—O₂R⁸), carbamoyl (—CONR⁸), alkylcarbonyl, alkylarylcarbonyl, arylcarbonyl, arylalkylcarbonyl, hydroxyl (—OH), amino (NR⁸), halogen, allyl, alkoxy (—OR⁸), S-alkyl or S-aryl groups, R⁸ representing a C₁ a C₁₀ alkyl or aryl group.

The compound of the present invention comprising a tropane derivative may be used, for example, in the diagnosis of Parkinson's disease.

One example of a compound according to the present invention may consist of a structure of formula (F) in which R¹, R³ and R⁴═H and R² is NR⁵R⁶, in which R⁵═H and R⁶ is chosen from:

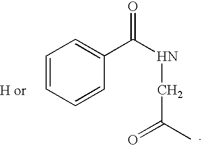

The compounds in which R₆=

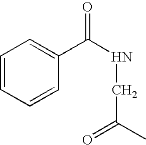

may be used, for example, for the diagnostic study of renal function or the therapy of renal pathologies.

The present invention also provides a chelation product consisting of a chelation compound according to the present invention, and of a metal or a metal complex. Examples of metals and metal complexes have been described above.

For example, the metal may be chosen from copper, a copper isotope and a transition metal. This may be useful, for example, as a radiopharmaceutical for therapy or diagnosis.

For example, the metal complex may be TcN or ReN. This product may then be used as a radiopharmaceutical.

The present invention also relates to the use of a chelation compound or a chelation product according to the present invention for manufacturing a medicinal product or a diagnostic product, for example a radiopharmaceutical for therapy or diagnosis. The radiopharmaceutical may be, for example, a radiopharmaceutical for visualizing the uptake of dopamine or serotonin. Such a radiopharmaceutical may be useful for diagnosing neurodegenerative diseases, for example Parkinson's disease.

The present invention also provides a process for manufacturing a bis-dithiocarbamate compound according to the invention, comprising, successively:

a step of protecting the XH functions of the compounds of formulae (V) and (VI) below:

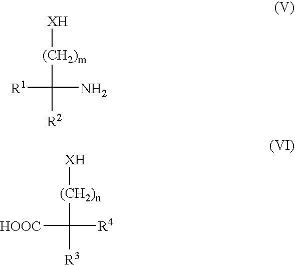

in which R¹, R², R³, R⁴, X, m and n are as defined in the above claim,

a step of activating the —COOH function of compound (VI),

a step of linking the compound of formula (V) and compound (VI) via the activated carboxyl function of compound (VI),

a step of deprotecting the XH functions, and

a step of reacting the deprotected XH functions with CS₂ to form a bis-dithiocarbamate compound according to the present invention.

One of the starting compounds comprises an acid function and the other an amine function to form the peptide bond which links these compounds. In addition, each of the compounds must comprise at least one free NH₂ or SH function to attach the CS₂.

The aim of the step for protecting the XH functions of compounds (V) and (VI) is to protect these functions against the reagents used to activate the —COOH function of compound (VI), and to link compound (V) with the activated compound (VI). It is performed by means of conventional reagents known to those skilled in the art for protecting functions X, with X═S or NH. Examples are given below.

The other steps may each also be performed by processes known to those skilled in the art. Examples are given below to illustrate the present invention.

The base of the chelation compound of the present invention may be, for example, a combination of two natural or unnatural amino acids containing two amine functions in ε-terminal positions and, on axial chains, a function which will serve to functionalize the vector molecule for use in radiopharmacy. The amine functions in ε-terminal positions are modified into dithiocarbamate functions et serve to complex the metal, also known as the metallic core.

The present invention also relates to a process for manufacturing a compound according to the present invention, the said process comprising a process for manufacturing a bis-dithiocarbamate compound according to the invention, and also comprising a step of attaching a radical R⁵ and optionally R⁶ to this bis-dithiocarbamide structure or to an intermediate product in its manufacture to obtain a chelation compound according to the invention as defined above.

The present invention thus provides a family of complexing agents which attach a metal, for example a radioelement, on the one hand, and which, by virtue of their functionalization, may be linked to a vector molecule either via a final synthesis (linking of radicals R⁵ or R⁶), or during the synthesis of a vector molecule or a peptide.

The compound for chelating a metal or a metal complex of the present invention may, for example, on the one hand, attach a radioelement, and, on the other hand, be linked to a vector molecule either via a final synthesis, or during the synthesis of a vector molecule or a peptide.

The present invention also relates to a process for manufacturing a compound according to the invention, the said process comprising:

a step of reacting two ε-NH₂ functions of two contiguous amino acids of a precursor molecule of the compound according to claim 1 or 2 with CS₂ so as to form a compound according to claim 1 or 2,

the precursor molecule constituting the radical R⁵ and optionally the radical R⁶.

The precursor molecule may be, for example, in the form of the compounds (V) and (VI) described above linked via a peptide bond formed between the NH₂ and COOH side functions.

The compound of the present invention may thus also be obtained, for example, by dithiocarbamate (DTC) labelling of two contiguous natural or unnatural amino acids, on the NH₂ functions of the side chains, or on an NH₂ function of a side chain and an N-terminal NH₂ function in the case of a dipeptide or of a reaction at the N-terminal end of a peptide or a protein. It may also be obtained by DTC-labelling of an organic molecule comprising a peptide bond and two amine functions separated by at least seven carbons.

The process of the present invention may be used, for example, to manufacture a chelation product according to the invention defined above, comprising the manufacture of a bis-dithiocarbamate compound according to the invention according to a manufacturing process of the present invention, and a reaction for complexing a metal or a metal complex via the said bis-dithiocarbamate compound manufactured.

The metal or the metal complex may be as defined above.

The present invention also provides a diagnostic kit comprising a chelating compound according to the present invention.

Other characteristics and advantages of the invention will also emerge on reading the examples which follow.

### EXAMPLES

A) Examples of the Preparation of Bis-Dithiocarbamates According to the Present Invention from Dipeptides

### Example 1

### Preparation of Bis-Dithiocarbamates from the Following Sequences: Lysine-Lysines (1), Alanine-Lysine (2) and Glycine-Lysine (3)

Each diamino molecule (0.5 mmol) is suspended in 10 ml of ethanol and sodium hydroxide pellets (0.08 g; 2 mmol) are added along with the minimum amount of water required to dissolve the amino molecule. The mixture is left stirring for 1 hour and the water/ethanol mixture is then evaporated off under reduced pressure at low temperature. The oil obtained is taken up in alcohol, 3 sodium hydroxide pellets are added and an excess of pure carbon disulphide CS₂ is then introduced dropwise. A yellow coloration appears after half an hour, and the copper sulphate test is positive. This test was performed by placing a drop of reaction mixture on a silica plate and then, on this drop, a drop of copper sulphate in water: a brown spot is observed if the bis-dithiocarbamate is formed. After stirring for 4 hours, the solvent is evaporated off to dryness, giving a yellow paste.

HPLC analysis: C18-5 μm-25 cm-YMC column; flow rate 1 ml/minute; UV detection at 300 nm; eluents A=water; B=methanol; gradient 0 to 5 minutes 0% of B—from 5 to 20 minutes 0 to 100% of B—from 20 to 25 minutes 100% of B; 25 to 25.1 minutes 100 to 0% of B—25.1 to 30 minutes 0% of B. Purity of the synthesized products greater than 95% in the three cases.

### Example 2

### Radiolabelling of the Lysine-Lysines (DTClys-lysDTC): Compound (4); Alanine-Lysine (DTCala-lysDTC): Compound (5) and Glycine-Lysine (DTCgly-lysDTC): Compound (6) Bis-Dithiocarbamates

Protocol for radiolabelling with ⁹⁹Tc:

a—intermediate solution: a lyophilisate containing hydrazine (SDH) 5 mg, 1,2-propanediamino-N,N,N′,N′-tetraacetic acid (PDTA) 5 mg, 10 μg of stannic chloride is taken up in 1 ml of injection-grade water. 1 ml of ⁹⁹TcO₄⁻ is added. The mixture is left to act for 30 minutes.

b—exchange reaction: 1.8 mg of bis-dithiocarbamate dissolved in a 0.1 M pH 7.4 phosphate buffer are mixed with 0.5 ml of the preceding intermediate solution. The mixture is left to act for 2 hours.

The reactions are analysed by HPLC. The results of these analyses are given in Table I below:

| Product | Detection | Number of peaks |
|---|---|---|
| (DTCgly-lysDTC) | radioactive | 9 peaks |
| (DTCala-lysDTC) | radioactive | 6 peaks |
| (DTClys-lysDTC) | radioactive | 1 peak |

The first two bis-dithiocarbamates react inter-molecularly whereas the (DTCgly-lysDTC) bis-dithiocarbamate reacts via an intramolecular reaction.

B) Examples of Steps for Protecting the XH Functions of Compounds of Formulae V and VI According to the Process of the Invention

### Example 3

### Synthesis of N-trifluoroacetyl-5-amino-valeric Acid: Compound (7)

1.6 equivalents of S-ethyl trifluorothioacetate (16 mmol; 2 ml) are added dropwise, using a syringe, to a solution of 5-aminovaleric acid (5a) (10 mmol; 1.17 g) dissolved in a mixture of 1N NaOH (16 mmol; 10 ml). [lacuna] in a three-necked flask fitted with a septum, through which is passed a flow of compressed air. A characteristic evolution of ethanethiol is observed.

The reaction medium is stirred for 24 hours at room temperature; a white precipitate forms. 1 ml of concentrated hydrochloric acid is then added. The precipitate is then filtered off on a sinter funnel and dried in the open air or in a desiccator.

The crude product is purified by recrystallization from 10 ml of a benzene/hexane mixture (1/1) to give 1.60 g of N-trifluoroacetyl-5-aminovaleric acid of formula (7) below:

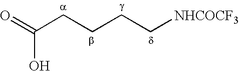

N-trifluoroacetyl-5-aminovaleric Acid (Compound 7)

¹H NMR (D₂O): 1.0 (m, 4H, H_{β}, H_{γ}); 2.2-2.3 (m, 2H, H_{δ}); 3.2 (m, 2H, H_{α})

¹³C NMR (D₂O): 26.5 (C_{β}); 30; 0 (C_{γ}); 34.1 (C_{α}); 39.9 (C_{δ}); 116.7 (CF₃, q, ¹J_{C-F}=285.9 Hz); 157.6 (COCF₃, q, ²J_{C-F}=36.6 Hz); 176.1 (COOH).

### Example 4

### Synthesis of N-trifluoroacetyl-6-amino-caproic Acid: Compound (8)

The process is performed in the same way as in Example 3, to give compound (8). The process is commenced using 1 equivalent of 6-aminocaproic acid (10 mmol; 1.31 g) dissolved in 1 equivalent of 1N NaOH (10 mmol; 10 ml), to which are added 1.6 equivalents of S-ethyl trifluorothioacetate (16 mmol; 2 ml). After purification, 1.70 g of N-trifluoroacetyl-6-amino-caproic acid (6b) (yield=75%) of formula 8 below are recovered:

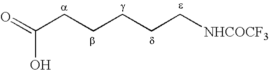

N-trifluoroacetyl-6-aminocaproic Acid (Compound 8),

¹H NMR (D₂O): 1.13-1.37 (m, 2H, H_{γ}); 1.45-1.54 (m, 4H, H_{β}, H_{δ}); 2.21 (t, 2H, ³J_{He-Hδ}=7.7 Hz, H_{ε}); 3.16-3.25 (m, 2H, H_{α}); 9.5 (s, 1H, NH).

¹³C NMR (acetone): 24.8 (C_{β}); 26.5 (C_{γ}); 30 (C_{δ}); 34.1 (C_{α}); 30.9 (C_{ε}); 116.7 (CF₃, q, ¹J_{C-F}=285.9 Hz); 157.6 (COCF₃, q, ²J_{C-F}=36.6 Hz); 176.1 (COOH).

### Example 5

### Synthesis of N-trifluoroacetylornithine: Compound (9)

The protocol is identical to that described above: 1.6 equivalents of EtSCOCF₃ (16 mmol; 2 ml) are added to a solution of ornithine monohydrochloride (10 mmol; 1.68 g or 1.82 g, respectively) in a mixture of 1 N NaOH (10 mmol; 10 ml) [lacuna].

The crude product is purified by recrystallization from 10 ml of a water/ethanol mixture (1/1) to give 1.60 g of N^{δ}-trifluoroacetylornithine of formula 9 below, in the form of a white powder, in a yield of 70%:

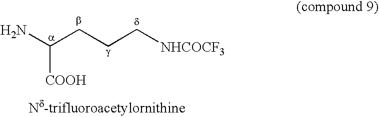

¹H NMR (D₂O): 1.5-2 (m, 4H, H_{β}, H_{γ}); 3.2-3.4 (m, 2H, H_{δ}), 4 (t, 1H, ³J_{Hα-Hβ}=6.1 Hz).

¹³C NMR (D₂O): 24.8 (C_{γ}); 28.2 (C_{β}); 39.6 (C_{δ}); 54-8 (C_{α}); 116.3 (CF₃, q, ¹J_{C-F}=285.2 Hz); 159 (COCF₃, q, ²J_{C-F}=³⁵ Hz); 174.9 (COOH).

### Example 6

### Synthesis of N-trifluoroacetyllysine: Compound (10)

The protocol is identical to that described above: 1.6 equivalents of EtSCOCF₃ (16 mmol; 2 ml) are added to a solution of lysine monohydrochloride (10 mmol; 1.82 g) in a mixture of 1 N NaOH (10 mmol; 10 ml) [lacuna].

The crude product is purified by recrystallization from 10 ml of a water/ethanol mixture (1/1) to give 1.70 g of N^{ε}-trifluoroacetyllysine of formula 10 below, in the form of a white powder, in a yield of 70%.

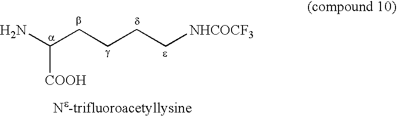

¹H NMR (D₂O): 1.2-1.3 (m, 2H, H_{δ}); 1.4-1.6 (m, 2H, H_{γ}), 1.7-1.8 (m, 2H, H_{β}); 3.2 (t, 2H, ³J_{Hε-Hδ}=6.5 Hz, H_{ε}); 3.6 (t, 1H, ³J_{Hα-Hβ}=6.2 Hz, H_{α}).

¹³C NMR (D₂O): 25.9 (C_{γ}); 28.5 (C_{δ}); 31.7 (C_{β}); 37.3 (C_{ε}); 46.8 (C_{α}); 113.7 (CF₃, q, ¹J_{C-F}=287.8 Hz); 155.2 (COCF₃, q, ²J_{C-F}=37.14 Hz); 167.3 (COOH).

### Example 7

### Synthesis of N^{ε}-trifluoroacetyldiaminopentane: Compound (22)

50 mmol of diaminopentane are dissolved in 100 ml of methanol. A solution of 50 mmol of ethyl trifluorothioacetate in 10 ml of methanol is added dropwise. The reaction mixture is stirred for 3 hours. After evaporating off the solvent, a yellow oil is obtained, which partially crystallizes in ice. This synthesis may be represented schematically in the following manner:

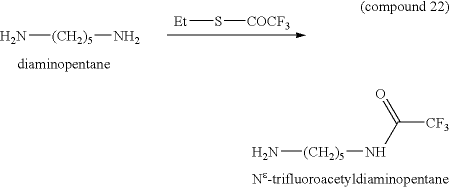

A purification is performed by flash chromatography on silica gel, with the eluent: CH₂Cl₂/MeOH (90/10).

I.R. (KBr): 3500-3400 cm⁻¹ (v NH amide and amine, broad band), 2867 cm⁻¹ (v CH₂ of the alkyl chain), 1711 cm⁻¹ (v CO of the amide), 1490 cm⁻¹ (v CH of s CH₂).

¹H NMR (DMSO D₆): 3.13 [t(J=7.05 Hz); 2H; CH₂α]; 2.46 [t(J-6.54 Hz); 2H; CH₂ε]; 1.43 [m(J≈7 Hz); 2H; CH₂β]; 1.24 [m; 4H; CH₂δ and γ].

¹³C NMR (DMSO): 156.1 [q; C=0 of the trifluoroacetamide]; 116 [q; CF₃]; 41.5, 39.2, 32.8, 28.2, 23.6 [s, 5 CH₂].

### Example 8

### Synthesis of N^{α}-Boc-diaminobutane: Compound (27)

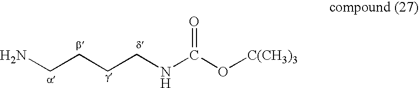

A solution of di-tert-butyl dicarbonate (4.9 g, 0.022 mol) in dioxane (60 ml) is added over a period of 2.5 hours to a solution of 1,4-butanediamine (15.51 g, 0.176 mol) in dioxane (60 ml). The mixture is stirred for 22 hours and the solvent is evaporated off on a rotavapour. Water (50 ml) is added to the residue and the insoluble disubstituted product is recovered by filtration. The filtrate is dried with anhydrous magnesium sulphate. Next, it is extracted with methylene chloride (3×50 ml). After evaporating off the solvent, 3.4 g of a colourless oil (compound 27) (81%) are obtained, and gradually solidified to give a white solid (m.p.=112° C., Lit*.m.p.=110-112° C.). *A. P. Krapcho, C. S. Kuell, Mono-protected diamines, N-tert-butoxycarbonyl-α-ω-alkanediamines From α,ω-Alkanediamines, Synthetic Communications, 1990, 20, 2559-2564.

¹H NMR (CDCL₃): δ(ppm)=1.28 (s, 2H, NH₂); 1.3-1.6 (m, 4H, H_{γ′}, H_{β′}); 1.45 (s, 9H); 2.65 (t, 2H, H_{α′}); 3.04 (q, 2H, H_{δ′}); 4.7 (sl, 1H).

¹³C NMR (50.2 MHz, CDCl₃/CHCl₃: 77 ppm/TMS): δ(ppm)=155.80 (COOC(CH₃)₃); 78.72 (C(CH₃)₃); 41.57 (C_{δ′}); 40.16 (C_{α′}); 30.62 (C_{γ′}); 28.16 ((CH₃)₃); 27.24 (C_{β′}).

C) Examples of Steps for Activating the COOH Functions of the Compounds of Formula VI According to the Process of the Invention

### Example 9

### Activation of the Acid Functions of Compounds (7) and (8)

470 mg of compound (7)—or 500 mg of compound (8)—(2.20 mmol) are dissolved in 20 ml of ethyl acetate in a 50 ml round-bottomed flask. 1 equivalent of N-hydroxysuccinimide (2.20 mmol; 253 mg) is added. 1 equivalent of dicyclohexylcarbodiimide (2.20 mmol, 454 mg) is added to this clear solution. The mixture is stirred for 24 hours at room temperature. A white precipitate of N,N′-dihexylurea appears rapidly, and is removed by filtration through a sinter funnel. The filtrate is recovered and evaporated. A pale yellow oil is obtained, which is taken up, if necessary, in a small amount of ethyl acetate, and the mixture is refiltered. This operation removes the remaining urea. The filtrate is evaporated to give a transparent oil which crystallizes at room temperature to give 682 mg of compound (11)—or 713 mg of compound (12)—in the form of a pearlescent white solid (yield=90%).

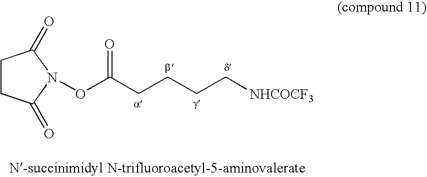

¹H-NMR (CDCl₃): 1.25-1.9 (m, 4H, H_{ε}, H_{γ}); 2.6 (t, 2H, ³J_{Hδ′-Hγ′}=6.3 Hz); 2.8 (s, 4H, H_{succinimide}); 3.2-3.4 (m, 2H, H_{α′}); 7.4 (s, 1H, NH).

¹³C NMR (CDCl₃): 22.0 (C_{β′}); 30.7 (C_{γ′}); 39.5 (C_{δ′}); 60.8 (C_{α′}); 119.1 (CF₃, q, ¹J_{C-F}=287.6 Hz); 157.5 (COCF₃, q, ²J_{C-F}=36.78 Hz); 168.7 and 169.9 (2 CO_{succinimide}); 171.8 (CO—O).

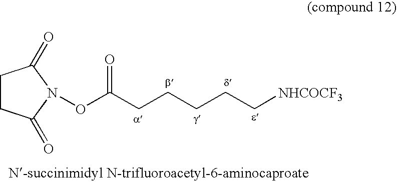

¹H-NMR (CDCl₃): 1.2-2.0 (m, 6H, H_{β}, H_{γ}, H_{δ}); 2.5 (t, 2H, ³J_{Hε-Hδ}=6.5 Hz); 2.8 (s, 4H, H_{succinimide}); 3.2-3.3 (m, 2H, H_{α}); 7.5 (s, 1H, NH).

¹³C NMR (CDCl₃): 21.4 (C_{γ}); 22.0 (C_{β}); 28.0 (C_{δ}); 39.6 (C_{ε}); 60.8 (C_{α}); 119.0 (CF₃, q, ¹J_{C-F}=289.3 Hz); 157.5 (COCF₃, q, ²J_{C-F}=36.7 Hz); 168.6 and 168.8 (2 CO_{succinimide}); 171.7 (CO—O).

### Example 10

### Activation of Hippuric Acid with N-hydroxysuccinimide: Compound (23)

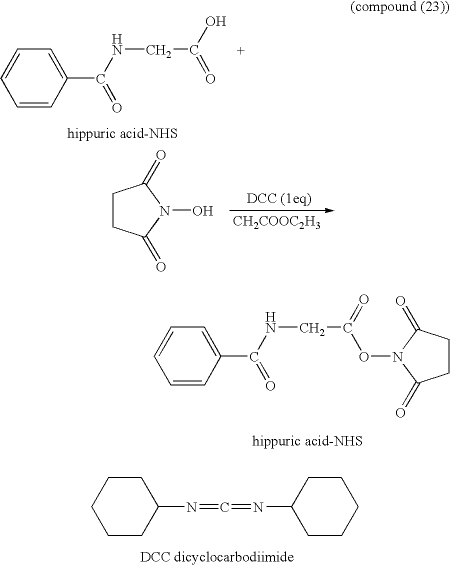

17.3 mmol (2 g) of N-hydroxysuccinimide are dissolved in 60 ml of ethyl acetate. 17.3 mmol (3.1 g) of hippuric acid are added. A solution of 17.3 mmol (3.57 g) of dicyclohexylcarbodiimide in 15 ml of ethyl acetate is then added. A voluminous white precipitate forms. The reaction mixture is stirred for 15 hours. The solution is filtered and a white solid is recovered.

Purification:

Cold fractional recrystallization from ethyl acetate.

m.p.=149-150° C.

I.R. (KBr): 3357 cm⁻¹ (strong band; v N—H of the amide), 1813, 1785, 1740 cm⁻¹ (v C=0 of the 4 carbonyl functions) 1640, 1579 cm⁻¹ (v of the aromatic rings).

¹H NMR (DMSO D₆): 9.2 [t; 1H; NH amide]; 7.9 [d; 2H; aromatic Hₒᵣₜₕₒ]; 7.5 [m; 3H; Hₘₑₜₐ and Hₚₐᵣₐ; 4.45 [d; 2H; aliphatic CH₂]; 2.8 [s; 4H; 2 cyclic CH₂].

### Example 11a)

### Coupling of Hippuric Acid and Lysine to Form Compound (24)

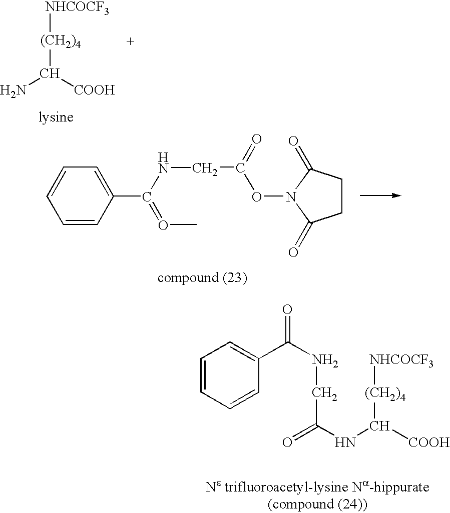

1.5 mmol (0.5 g) of compound 23 are dissolved in 20 ml of tetrahydrofuran. 1.5 mmol (0.4 g) of N^{ε}-trifluoroacetyllysine and 1.5 mmol (0.2 ml) of triethylamine are then added and, after stirring for 20 hours, the mixture is clear. The THF is evaporated off and the oil obtained is taken up in ethyl acetate and washed with water. The aqueous phase is acidified with a few drops of concentrated acetic acid and then extracted with dichloromethane. A white solid precipitates in the organic phase (product).

¹H NMR (DMSO D₆): 9.4 [t; 1H, NH of the hippuric acid]; 8.7 [t; 1H; NH trifluoroacetamide]; 8.2 [d; 1H; amide NH of the coupling]; 7.8 [d; 2H, Hₒᵣₜₕₒ]; 7.5 [m; 3H; Hₚₐᵣₐ and 2 Hₘₑₜₐ]; 3.9 [t; 2H; CH₂ of the hippuric acid]; 3.1 [m; 2H; CH₂ε]; 1.1-1.8 [3 m; 6H; 3 CH₂ of the aliphatic chain].

### Example 11 b)

### Activation of N^{ε}-trifluoroacetyllysine N^{α}-hippurate: (Compound 24) with N-hydroxysuccinimide to Form Compound (25)

10 mmol (2 g) of N-hydroxysuccinimide are dissolved in 60 ml of ethyl acetate. 10 mmol of N^{ε}-trifluoroacetyllysine N^{α}-hippurate are added. A solution of 10 mmol of dicyclohexylcarbodiimide in 20 ml of ethyl acetate is added. The reaction mixture is stirred for 24 hours. The solution is filtered and a white solid is recovered.

¹H NMR (DMSO D₆): 9.4 [t; 1H, NH of the hippuric acid]; 8.7 [t; 1H; NH trifluoroacetamide]; 8.2 [d; 1H; amide NH of the coupling]; 7.8 [d; 2H, Hₒᵣₜₕₒ]; 7-5 [m; 3H; Hₚₐᵣₐ and 2 Hₘₑₜₐ]; 3.9 [t; 2H; CH₂ of the hippuric acid]; 3.1 [m; 2H; CH₂ε]; 2.8 [s; 4H; 2 CH₂]; 1.1-1.8 [3 m; 6H; 3 CH₂ of the aliphatic chain].

Purification:

Cold fractional recrystallization from ethyl acetate.

D) Examples of Steps for Linking a Compound of Formula V and a Compound of Formula VI Activated via its Carboxyl Function According to the Process of the Invention

### Example 12

### Synthesis of N^{δ},N^{δ}-bis(trifluoroacetyl)-N-(5-aminopentanoyl)ornithine: Compound (13)

1 equivalent of compound (9) or (lacuna] (2 mmol; i.e. 456 mg) is dissolved in 10 ml of dimethylformamide in a 25 ml round-bottomed flask. A suspension is obtained, to which is added 1 ml of triethylamine and 1.5 equivalents of compound (11) (3 mmol; 639 mg or 681 mg, respectively). The reaction mixture is stirred for 24 hours at room temperature to give a clear solution.

The mixture is then made alkaline by successive additions of a sodium carbonate solution until a pH=8 is obtained. Next, the mixture is extracted with twice 15 ml of ethyl acetate. The aqueous phase is recovered and hydrolysed with a few ml of concentrated hydrochloric acid until a pH=2 is obtained. The resulting mixture is extracted with three times 15 ml of ethyl acetate. The organic phases are then washed with water to remove the remaining DMF, and then with brine and are dried over Na₂SO₄. The solvent is then evaporated off to give a clear oil which crystallizes at room temperature. The yield is 70%.

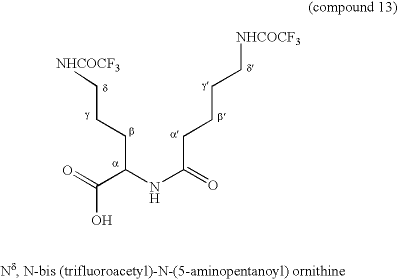

¹H NMR (acetone): 1.5-2.2 (2m, 8H, H_{β}H_{β′}, H_{γ},H_{γ′}); 2.3 (m, 2H, H_{α′}); 3.4 (t, 4H, H_{δ}H_{δ′}, ³J_{Hδ-Hγ}=6 Hz, ³J_{Hδ′-Hγ′}=6 Hz); 4.5 (m, 1H, H_{α}); 7.6 (d, 1H, ³J_{NH-Hα}=7.6 Hz); 8.6 (s 2H, 2NHCOCF₃)—

¹³C NMR (DMSO d₆): 23.3 to 29.2 (C_{β}C_{β′}C_{γ}C_{γ′}); 36.6 (C_{α′}); 41.1 and 41.9 (C_{δ}C_{δ′}); 52.3 (C_{α}); 116.8 (CF₃, q, ¹J_{C-F}=288.3 Hz); 157.0 (COCF₃, q, ²J_{C-F}=36.7 Hz); 172.9 (CONH); 174.5 (COOH).

### Example 13

### Synthesis of N^{δ},N-bis(trifluoroacetyl)-N-(5-aminohexanoyl)ornithine: Compound (14)

The process is performed in the same manner as in Example 12 with 1 equivalent of compound (9) (2 mmol; i.e. 456 mg). Compound (11) is replaced with 1.5 equivalents of compound (12) (3 mmol; i.e. 681 mg). The yield is 70%.

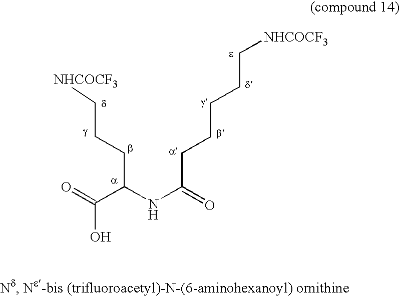

¹H NMR (DMSO d₆): 1.1-1.8 (m, 10H, H_{β}H_{β′}, H_{γ}H_{γ′}H_{δ′}); 2.1 (t, 2H, ³J_{Hα′-Hβ′}=7.2 Hz, H_{α′}); 3.2 (m, 4H, H_{ε′}H_{δ}); 4.1 (m, 1H, H_{α}); 8.1 (d, 1H, ³J_{NH-Hα}=7.7 Hz, NH); 9.4 (s, 2H, 2NHCOCF₃).

¹³C NMR (DMSO d₆): 27.4 to 30.9 (C_{β}C_{β′}C_{γ}C_{γ′}C_{δ′}); 37 (C_{α′}); 41 (C_{ε′}C_{δ}); 54 (C_{α}); 120 (2 CF₃, ¹J_{C-F}=288.2 Hz); 158.6 (2 COCF₃, q, ²J_{C-F}=39.0 Hz); 174.9 (CONH); 176.2 (COOH).

### Example 14

### Synthesis of N^{δ},N^{δ}-bis(trifluoroacetyl)-N-(5-aminopentanoyl)lysine: Compound (15)

1 equivalent of compound (10) (2 mmol; i.e. 484 mg) is dissolved in 10 ml of dimethylformamide in a 25 ml round-bottomed flask. A suspension is obtained, to which is added 1 ml de triethylamine and 1.5 equivalents of compound (11) (3 mmol; i.e. 639 mg or 681 mg). The reaction mixture is stirred for 24 hours at room temperature to give a clear solution.

The mixture is then made alkaline by successive additions of a sodium carbonate solution until a pH=8 is obtained. Next, the mixture is extracted with twice 15 ml of ethyl acetate. The aqueous phase is recovered and hydrolysed with a few ml of concentrated hydrochloric acid until a pH=2 is obtained. The resulting mixture is extracted with three times 15 ml of ethyl acetate. The organic phases are then washed with water to remove the remaining DMF, and then with brine and are dried over Na₂SO₄. The solvent is then evaporated off to give a clear oil which crystallizes at room temperature. The yield is 70%.

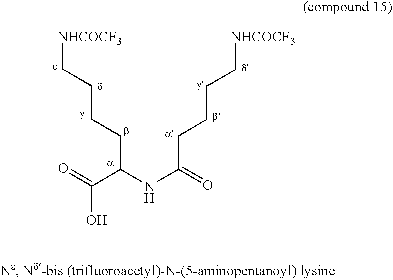

¹H NMR (200 MHz, acetone): 1.1-1.8 (m, 5H, H_{β}H_{β′}, H_{γ}H_{γ′}); 2.2 (t, 2H, ³J_{Hα′-Hβ′}=6.5 Hz, H_{α′}); 3.2 (m, 4H, H_{ε′}H_{δ′}); 4.3 (m, 1H, H_{α}); 7.5 (d, 1H, ³J_{NH-Hα}=7.8 Hz, NH); 8.4 (s, 2H, 2NHCOCF₃).

¹³C NMR (acetone): 23 to 31 (C_{β}C_{β′}C_{γ}C_{γ′} and C_{δ}); 39 (C_{α′}); 49 (C_{ε}C_{δ′}); 52 (C_{α}); 117 (2 CF₃, ¹J_{C-F}=287.4 Hz); 156 (2 COCF₃, q, ²J_{C-F}=35.3 Hz); 173.5 (CONH); 173.7 (COOH).

### Example 15

### Synthesis of N^{δ},N^{δ}-bis(trifluoroacetyl)-N-(5-aminohexanoyl)lysine: Compound (16)

The process is performed in the same manner as in Example 14, with 1 equivalent of compound (10) (2 mmol; i.e. 456 mg or 484 mg). Compound (11) is replaced with 1.5 equivalents of compound (12) obtained in Example 9 above (3 mmol; i.e. 681 mg). The yield is 70%.

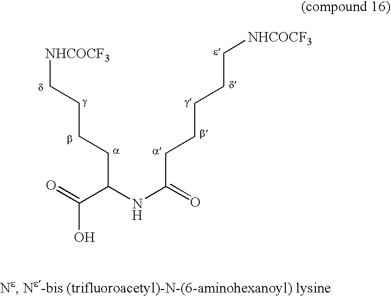

¹H NMR (DMSO d₆): 1.2-1.7 (3m, 12H, H_{β}H_{β′}H_{γ}H_{γ′}H_{δ}H_{δ′}); 2.1 (t, 2H, ³J_{Hα′-Hβ′}=7.3 Hz, H_{α′}); 3.1-3.25 (m, 4H, H_{ε′}H_{ε′}); 4.0-4.2 (m, 1H, H_{α}); 8.1 (d, 1H, ³J_{NH-Hα}=7.7 Hz, NH); 9.5 (s, 2H, 2NHCOCF₃); 12.3 (s, 1H, COOH).

¹³C NMR (DMSO d₆): 23.5-34.3 (C_{β}C_{β′}C_{γ}C_{γ′}C_{δ}C_{δ′}); 36.6 (C_{α′}); 116.8 (2 CF₃, q, ¹J_{C-F}=2870.8 Hz); 156.8 (2 COCF₃, q, ²J_{C-F}=35.7 Hz); 173.1 (CONH); 174.6 (COOH).

### Example 17

### Coupling of N^{ε}-trifluoroacetyllysine N^{α}-hippurate NHS (24) to N^{ε}-trifluoroacetyldiaminopentane (22) to Form Compound (26)

1.5 mmol of N^{ε}-trifluoroacetyllysine N^{α}-hippurate NHS (24) are dissolved in 50 ml of tetrahydrofuran. 1.5 mmol of N^{ε}-trifluoroacetyldiaminopentane (22) and 1.5 mmol (0.2 ml) of triethylamine are then added and, after stirring for 20 hours, the mixture is clear. The THF is evaporated off and the oil obtained is taken up in ethyl acetate and washed with water. The aqueous phase is acidified with a few drops of concentrated acetic acid and then extracted with dichloromethane. A white solid precipitates in the organic phase.

This reaction may be represented schematically in the following manner:

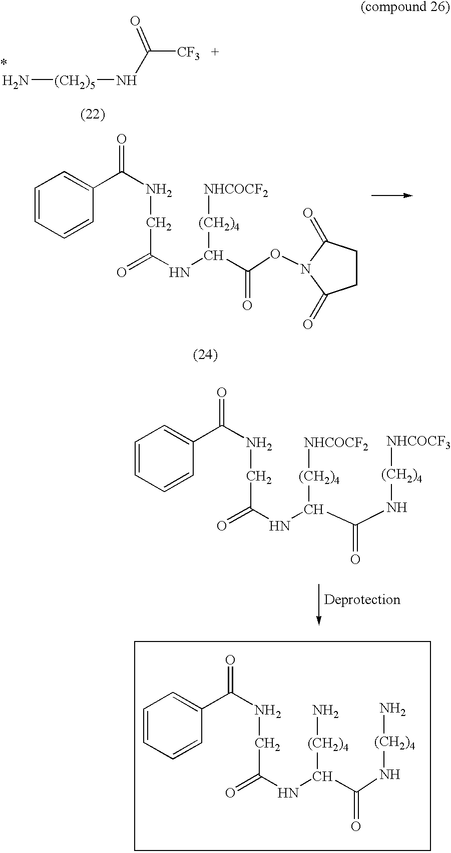

¹H NMR (DMSO D₆): 9.4 [t; 1H, NH of the hippuric acid]; 8.7 [t; 1H; NH trifluoroacetamide]; 8.2 [d; 1H; amide NH of the coupling]; 7.8 [d; 2H, Hₒᵣₜₕₒ]; 7.5 [m; 3H; Hₚₐᵣₐand 2 Hₘₑₜₐ]; 3.9 [t; 2H; CH₂ of the hippuric acid]; 3.1-3.2 [m; 4H]; 2.4-2.5 [t; 2]; 1.1-1.8 [m; 12H; 6 CH₂ of the aliphatic chains].

### Example 18

### Synthesis of N^{ε},N^{δ′}-bis(tert-butoxycarbonyl)-(α′,δ′-diaminobutyl)-N^{α}(carboxybenzyloxy)-D-lysine: Compound (28)

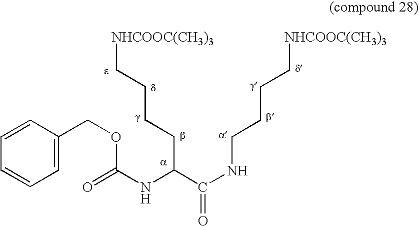

0.200 g (1.067×10⁻³ mol) of N-tert-butoxycarbonyl-1,4-diaminobutane (compound (27)), 0.405 g (1.067×10⁻³ mol) of N^{α}-CBz-N^{ε}-tBoc-D-lysine, 0.220 g (1.067×10⁻³ mol) of 1,3-dicyclohexylcarbodiimide and 0.144 g (1.067×10⁻³ mol) of 1H-hydroxybenzotriazole are dissolved in 30 ml of dry dichloromethane in a 50 ml round-bottomed flask. A white precipitate of N,N′-dicyclohexylurea appears during the reaction. The reaction mixture is stirred for 20 hours at room temperature. The filtrate is evaporated to give a white solid, which is dissolved in H₂O (20 ml) and treated with a saturated NaHCO₃ solution (20 ml). The aqueous phase is extracted with dichloromethane (40 ml) and washed with water (10 ml). The organic phase is dried over MgSO₄ and evaporated to give a solid compound. The product is purified by flash chromatography (using various eluents: CH₂Cl₂ alone (100 ml) followed by EtOAc (200 ml). The pure fractions lead to 0.302 g, 52%, of solid product (compound (28)), m.p.=60-62° C.

¹H NM (200.13 MHz, CDCl₃/CHCl₃): 7.24 ppm/TMS): δ(ppm)=7.3 (m, 5H, Ph—H); 6.4 (s, 1H, NHCOO); 5.5 (sl, 1H, NHCO); 5.07 (m, 1H, Ph—CH₂O); 4.07 (m, 1H, H_{α}); 3.24 (m, 1H, H_{α′}); 3.06 (m, H_{ε}, H_{δ′}); 1.40 (m, 9H, (CH₃)₃); 1.87-1.23 (m, 5H, H_{β}, H_{β′}, H_{δ}, H_{γ}, H_{γ′}).

¹³C NMR (50.2 MHz, CDCl₃/CHCl₃: 77 ppm/TMS): δ(ppm)=171.57 (NHCO): 156.09 & 155.99 (2×COOC(CH₃)₃); 136.01 (aromatic C); 128.32 (aromatic CH₂); 128.00 (aromatic CH); 127.90 (aromatic CH₂); 79.13 (2×C(CH₃)₃); 66.81 (Ph—CH₂); 54.72 (C_{α}); 39.94 (C_{δ′}); 39.75 (C_{ε}); 31.90 (C_{δ}); 29.41 (C_{γ′}); 28.19 ((CH₃)₃); 27.36 (C_{γ}); 26.15 (C_{β′}); 22.26 (C_{β}).

Infrared

1689 cm⁻¹: v(NHCOO); 1652 cm⁻¹: v(NHCO).

Mass Spectrum

The molar mass is 550.

The peak 573 corresponds to (M′+Na).

Synthesis of N^{ε},N^{δ′}-bis(tert-butoxycarbonyl)-(α′,δ′-diaminobutyl)-D-lysine: Compound (29)

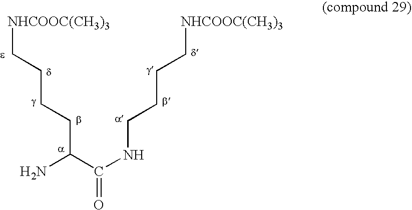

0.300 g (5.49×10⁻⁴ mol) of N^{ε},N^{δ′}-bis(tert-butoxycarbonyl)-(α′,δ′-diaminobutyl)-N^{α}(carboxybenzyloxy)-D-lysine (compound (28)) is dissolved in 20 ml of methanol in a 50 ml round-bottomed flask, and 40 mg of Pd/C (10%) are added. The reaction mixture is stirred for 60 hours; the Pd is removed by filtration using Celite. The solvent is removed under vacuum to give a colourless liquid. This liquid, washed with hexane and dried, gives 0.205 g (90%) of compound (29) (liquid).

¹H NMR (200.13 MHz, CDCl₃/CHCl₃: 7.24 ppm/TMS): δ(ppm)=4.76 (m, 1H, H_{α}); 3.18 (m, 1H, H_{α}); 3.04 (m, 4H, H_{ε}, H_{δ′}); 1.37 (m, 9H, (CH₃)₃); 1.68-1.37 (m, 5H, H_{β}, H_{β′}, H_{ε}, H_{γ}, H_{γ′}).

¹³C MM (50.2 MHz, CDCl₃/CHCl₃: 77 ppm/TMS): δ(ppm)=174.18 (NHCO): 155.89 (2×COOC(CH₃)₃); 78.85 (2×C(CH₃)₃); 54.62 (C_{α}); 39.91 (C_{δ′}); 38.55 (C_{ε}); 34.03 (C_{α}); 32.57 (C_{δ}); 29.60 (C_{γ′}); 28.19 ((CH₃) 3); 27.27 (C_{γ}); 26.60 (C_{β′}); 22.54 (C_{β}).

E) Example of a Process for Manufacturing a Bis-Dithiocarbamate Compound According to the Present Invention in Which a Radical R is Attached to an Intermediate Product of this Compound

### Example 19

### Synthesis of methyl N^{δ},N^{δ}-bis(trifluoroacetyl)-N-(5-aminohexanoyl)lysinate: Compound (17)

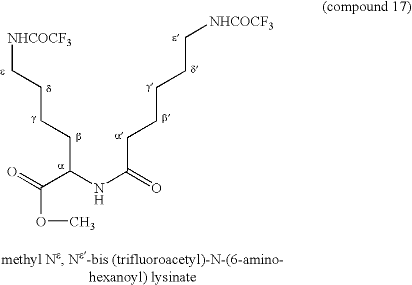

100 mg (0.2 mmol) of compound (16) are dissolved in 10 ml of absolute methanol. 2 equivalents of TMCS (0.4 mmol; 56 μl) are added slowly to this suspension and the mixture is stirred for 24 hours at room temperature. The solvent is evaporated off to give a yellow oil, which is dissolved in 20 ml of ethyl acetate. The organic phase is washed with 20 ml of a sodium carbonate solution and then with 20 ml of brine and dried over Na₂SO₄. The solvent is evaporated off to give 90 mg of methyl of N^{ε}N^{ε′}-bis (trifluoroacetyl)-N-(6-aminohexanoyl)lysinate (compound (17)) in a yield of 90%.

¹H NMR (CDCl₃): 1.25-2.0 (m, 6H, Hp, H_{β}, H_{β′}, H_{γ}, H_{γ′}, H_{δ}, H_{δ′}); 2.6 (m, 2H, H_{α′}); 3.4 (m, 4H, H_{ε}, H_{ε′}); 3.7 (s, 3H, CH₃); 4.6 (m, 1H, H_{α}); 6.5 (d, 1H, ³J_{NH-Hα}=7.1 Hz); 7.4 (s, 2H, 2 NHCOCF₃).

IR (cm⁻¹): 3104, 3238, 3417, 3480 (NHCOCF₃ and —NH—), 1743 (C=0 ester).

### Example 20

### Synthesis of 2β-[N^{δ},N^{δ}-bis(trifluoroacetyl)-N-(5-aminopentanoyl)ornithyloxymethyl]-3β-(4′-tolyl)tropane: Compound (18)

0.8 equivalent of compound (13) obtained in Example 12 above (0.65 mmol, i.e. 275 mg) is added to a solution of 2β-hydroxymethyl-3β-(4′-tolyl)tropane (200 mg; 0.8 mmol) stirred at room temperature and under an inert atmosphere, for example nitrogen, in 20 ml of dichloromethane.

0.8 equivalent of DMAP (0.65 mmol; 80 mg) and 0.8 equivalent of EDCI (0.65 mmol, 125 mg) are then added. After stirring for 15 hours, the reaction medium is washed with an NaHCO₃ solution (20 ml) and then with 1N hydrochloric acid solution (20 ml) and finally with brine (20 ml), and the organic phases are dried over Na₂SO₄ and then filtered. The solvent is evaporated off and a yellow oil is recovered in a yield of 50%.

¹H NMR (CDCl₃): 1.3-1.5 (m, 9H, H₂, H_{β}, H_{β′}, H_{γ}, H_{γ′}); 1.5-1.9 (m, 3H, H_{4α}, H_{6α}, H_{7α}); 2.0-2.1 (m, 4H, H_{6β}, H_{7β}, H_{α′}); 2.2 (s, 3H, pH-CH₃); 2.3 (s, 3H, N—CH₃); 3.0-3.6 (m, 8H, H₁, H₃, H₅, H_{4β}, H_{δ}, H_{δ′}); 3.8-4.0 (m, 1H, H_{α}); 4.3-4.5 (m, 2H, H₈); 6.25 (m, 1H, NH); 7.1-7.2 (m, 4H, aromatic H); 7.5 (m, 2H, 2 NHCOCF₃).

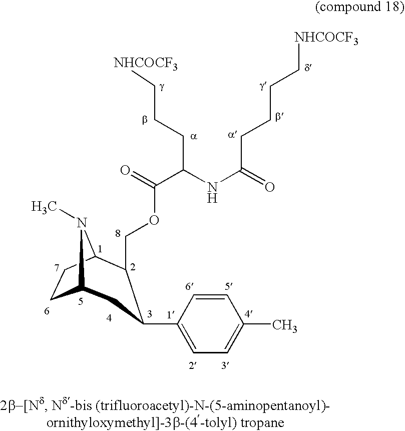

¹³C NMR (CDCl₃): 21.2 (Ph—CH₃); 22.6 and 25.0 (C_{β}and C_{β′}); 25.2 (C₆); 26.3 (C₇); 28.5 and 28.6 (C_{γ} and C_{γ′}); 34.2 (C_{α′}); 34.7 (C₃); 36.2 (C₄); 39.8 and 40.0 (C_{δ} et C_{δ′}); 42.3 (NCH₃); 45.7 (C₂); 52.1 (C_{α}); 62.3 (C₅); 64.1 (C₈); 65.6 (C₁); 116.4 (CF₃, q, ¹J_{C-F}=287.4 Hz); 127.8 (C_{2′} and C_{6′}); 129.4 (C_{3′}, C_{5′}); 136.2 (C_{1′}); 139 (C_{4′}); 157.6 (2 COCF₃, q, ²J_{C-F}=29.3 Hz); 172.5 (CONH); 173.4 and 173.6 (CO-0)-resolved signal.

### Example 21

### Synthesis of 2β-[N^{ε},N^{ε}-bis(trifluoroacetyl)-N-(5-aminohexanoyl)lysinyloxymethyl]-3β-(4′-tolyl)tropane: Compound (19)

The process is performed in the same manner as in Example 20, with a solution of 2β-hydroxymethyl-3β-(4′-tolyl)tropane (200 mg; 0.8 mmol). Compound (13) is replaced with 0.8 equivalent of compound (16) obtained in Example 15 above, (0.65 mmol; i.e. 284 mg). The solvent is evaporated off and a yellow oil is recovered in a yield of 50%.

¹H NMR (CDCl₃): 1.1-1.4 (m, 13H, H₂, H_{β}, H_{β′}, H_{γ}, H_{γ′}, H_{δ}, H_{δ′}); 1.4-1.8 (m, 3H, H_{4α}, H_{6α}, H_{7α}); 1.9-2.1 (m, 4H, H₆β, H_{7β}, H_{α′}); 2.2 (s, 3H, ph-CH₃); 2.3 (s, 3H, N—CH₃); 3.0-3.4 (m, 8H, H₁, H₃, H₅, H_{4β}, H_{ε}, H_{ε′}); 3.6-3.85 (m, 2H, H₈); 6.2 (m, 1H, NH), 7.1 (m, 4H, aromatic H); 7.5 (m, 2H, 2 NHCOCF₃).

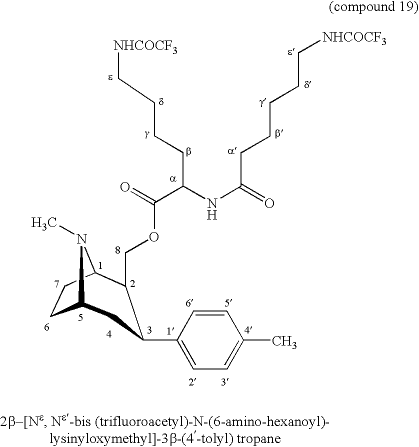

### Example 22

### Synthesis of 2β-[N^{δ},N^{ε}-bis(trifluoroacetyl)-N-(5-aminohexanoyl)ornithyloxymethyl]-3β-(4′-tolyl)tropane: Compound (20)

The process is performed in the same manner as in Example 20, with a solution of 2β-hydroxymethyl-3β-(4′-tolyl)tropane (200 mg; 0.8 mmol). Compound (13) is replaced with 0.8 equivalent of compound (14) obtained in Example 13 above (0.65 mmol; i.e. 284 mg). The solvent is evaporated off and a yellow oil is recovered in a yield of 50%.

¹H NMR (CDCl₃): 1.2-1.4 (m, 11H, H₂, H_{β}, H_{β′}, H_{γ}, H_{γ′}, H_{δ}); 1.4-1.9 (m, 3H, H_{4α}, H_{6α}, H_{7α}); 1.9-2.1 (m, 4H, H_{6β}, H_{7β}, H_{α′}); 2.2 (s, 3H, ph-CH₃); 2.3 (s, 3H, N—CH₃); 3.0-3.5 (m, 8H, H₁, H₃, H₅, H_{4β}, H_{δ}, H_{ε′}); 3.6-3.9 (m, 1H, H₈); 4.2-4.5 (m, 2H, H₈); 6.25 (m, 1H, NH); 7.0 (m, 4H, aromatic H); 7.4 (m, 2H, 2 NHCOCF₃).

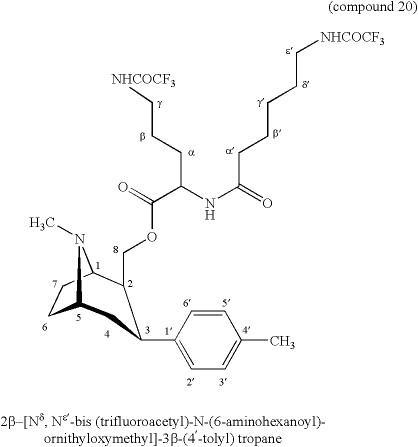

### Example 23

### Synthesis of 2β-[N^{δ},N^{ε}-bis(trifluoroacetyl)-N-(5-aminopentanoyl)lysinyloxymethyl]-3β-(4′-tolyl)tropane: Compound (21)

The process is performed in the same manner as in Example 20 with a solution of 2β-hydroxymethyl-3β-(4′-tolyl)tropane (200 mg; 0.8 mmol). Compound (13) is replaced with 0.8 equivalent of compound (15) obtained in Example 14 above (0.65 mmol; i.e. 293 mg). The solvent is evaporated off and a yellow oil is recovered in a yield of 50%.

¹H NMR (CDCl₃): 1.1-1.4 (m, 11H, H₂, H_{β}, H_{β′}, H_{γ}, H_{γ′}); 1.4-1.8 (m, 3H, H_{4α}, H_{6α}, H_{7α}); 2.0-2.1 (m, 4H, H_{6β}, H_{7β}, H_{α′}); 2.2 (s, 3H, ph-CH₃); 2.3 (s, 3H, N—CH₃); 2.7-3.9 (m, 1H, H_{α}); 4.2-4.5 (m, 2H, H₈); 4.2-4.5 (m, 2H, H₈); 6.2 (m, 1H, NH); 7.0 (m, 4H, aromatic H); 7.4 (m, 2H, 2 NHCOCF₃).

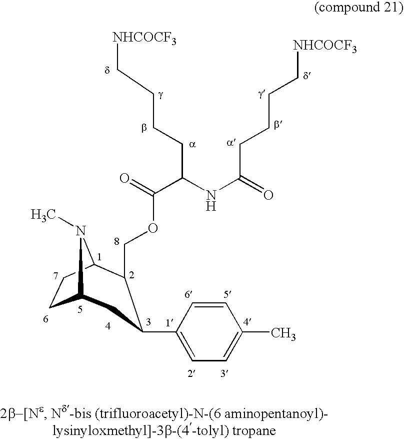

### Example 24

### 2β-[N^{ε},N^{δ′}-bis(tert-butoxycarbonyl)-(α′,δ′-diaminobutyl)-D-lysine]-3β-(4-tolyl)tropane: Compound (30)

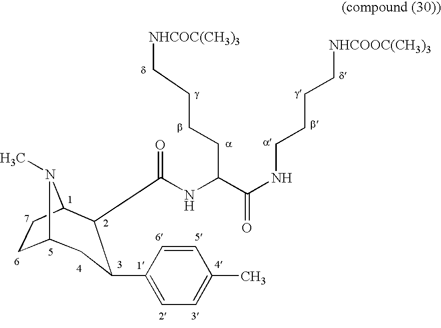

0.173 g (4.15×10⁻⁴ mol), of N^{ε},N^{δ′}-bis(tert-butoxycarbonyl)-(α′,δ′-diaminobutyl)-D-lysine (compound 29), 0.085 g (4.15×10⁻⁴ mol) of 1,3-dicyclohexylcarbodiimide, 0.056 g (4.15×10⁻⁴ mol) of 1H-hydroxybenzotriazole and 0.123 g (4.15×10⁻⁴ mol) of 2-β-carboxy-3β-tolyltropane are dissolved in 30 ml of dry dichloromethane in a 50 ml round-bottomed flask. A white precipitate of N,N′-dicyclohexylurea appears during the reaction. The reaction mixture is stirred for 20 hours at room temperature. The filtrate, recovered and evaporated, gives a white solid which is dissolved in water H₂O (10 ml) and treated with saturated NaHCO₃ solution (10 ml). The aqueous phase is extracted with dichloromethane (3×20 ml) and washed with water (10 ml); the organic phase obtained is dried over MgSO₄ and evaporated to give a liquid compound. The product is purified by flash chromatography (eluent: EtOAc (300 ml)). 0.09 g, 33%, of a colourless liquid product (compound 30) is obtained.

¹H NMR (200.13 MHz, CDCl₃/CHCl₃: 7.24 ppm/TMS): δ(ppm)=8.6 (m, 2H, NHCO); 7.23-7.6 (m, 4H, Ph—H); 6.2 (m, 1H, NH); 4.23 (m, 2H, H₈); 2.9-3.2 (m, 3H, H_{α}, H_{ε}, H_{δ′}); 2.1 (s, 3H, N—CH₃); 2.3 (s, 3H, Ph—CH₃); 1.89 (m, 4H, H6_{β}, H7_{β}, H_{α′}); 1.3-1.6 (m, 3H, H4_{α}, H6_{α}, H7_{α}); 1.35 (m, 9H, (CH₃)₃); 1.03-1.21 (m, 11H, H₂, H_{β}, H_{β′}, H_{δ}, H_{γ}, H_{γ′}).

¹³C NMR (50.2 MHz, CDCl₃/CHCl₃: 77 ppm/TMS): δ(ppm)=169.6 (NHCO): 168.03 (NHCO); 156.73 (2×COOC(CH₃)₃); 141.76 (C_{4′}); 128.42 (C_{4′}); 126.22 and 125.90 (C_{3′}, C_{5′}); 117.86 (C_{3′}); 79.43 (2×C(CH₃)₃); 75.79 (C4); 62.07 (C5); 54.03 (C_{α}); 49.59 (C₂); 41.55 (NHCHH₃)₃); 40.40 (_{cδ′}); 39.70 (_{cε}); 34.11 (C4); 31.43 (C_{ε}); 29.99 (C_{γ}); 29.64 ((CH3)₃); 28.78 (C6); 26.52 (C_{β′}); 22.54 (C_{β}); 22.40 (Ph—CH₃).

Infrared

1689 cm⁻¹: v(NHCOO); 1678 cm⁻¹: v(NHCO)

Mass Spectrum

The molar mass 4 is 657.

Peak 658 corresponds to (M+H).

F) Examples of Steps for Deprotecting the XH Functions and for Reacting These Deprotected Functions with CS₂ to Form a Bis-Dithiocarbamate Structure According to the Process of the Present Invention

### Example 25

### Synthesis of N^{ε},N^{δ′}-bis-dithiocarbamate)-(α′,δ-diaminobutyl)-N^{α}(carboxybenzyloxy)-D-lysine: Compound (31)

5 mg of compound (28) are dissolved in 500 μl of absolute methanol. 100 μl of trifluoroacetic acid are added. The mixture is stirred for 30 minutes. The mixture is evaporated under vacuum. When the reaction mixture is dry, 500 μl of methanol and 500 μl of piperidine are added. The mixture is left for a further 30 minutes. The reaction mixture is then evaporated to dryness under vacuum. 1 ml of methanol and 200 ml of carbon sulphide (CS₂) are then added. The reaction mixture is stirred at room temperature for 2 hours and then evaporated to dryness. Compound (31) is maintained dry at −18° C.

### Example 26

### 2β-[N^{ε},N^{δ′}-bis(dithiocarbamate)-(α′,δ′-diaminobutyl)-D-lysine]-3β-(4′-tolyl)tropane: Compound (32)

The process is performed in the same manner as Example 25, but with 5 mg of compound (30). The reaction mixture is stirred at room temperature for 2 hours and then evaporated to dryness. Compound (32) obtained is maintained dry at −18° C.

### Example 27

### The Deprotection and Synthesis of the Bis-Dithiocarbamates (Non-Functionalized Complexing Agents) of Products (13), (14), (15), (16) and (17) Obtained in the Preceding Examples Leads to the Corresponding Compounds (34), (35), (36), (37) et (38)

10 mmol of each peptide are dissolved in 5 ml of absolute methanol. 5 ml of a 0.1M solution of piperidine in methanol are added and the mixture is stirred for 1 hour. The mixture is evaporated under vacuum.

The dry residue is taken up in 5 ml of methanol and 3 ml of carbon sulphide are added. The mixture is stirred for 2 hours. The reaction mixture is evaporated to dryness and is stored at −18° C.

### Example 28

### Deprotection and Synthesis of the Bis-Dithiocarbamates (Substituted with a Tropane Derivative) of Products (18), (19), (20) and (21) Obtained in the Preceding Examples Leads to Compounds (44), (45), (46) and (47)

The process is performed in the same manner as in Example 27, but with the products (18), (19), (20) and (21). Compounds (44), (45), (46) and (47) are obtained.

G) Radiolabelling of Compounds According to the Invention

### Example 29

### The Radiolabelling of Products (31), (34) (35), (36), (37) and (38) with TcN Leads to Compounds (33), (39), (40), (41), (42) and (43)

Synthesis of the TcN intermediate 100 μg of tin chloride, 5 mg of SDH (succinyl dihydrazide) and 5 mg of PDTA (1,2-propanediamino-N,N,N′,N′-tetraacetic acid) were freeze-dried in a labelling flask. 3 ml of TcO₄⁻ (60 mCi) are added to this lyophilizate. The mixture is left to act for 15 minutes.

Complexation:

2 mg of bis-dithiocarbamate in 1 ml of ethanol are added to 1 ml of TcN. The mixture is left to react for one hour. The reaction is analysed by HPLC (reverse-phase, methanol-water). Labelling yield>95%.

The radiolabelling of compound 38 showed that the radiolabelled molecule, isolated by HPLC and left at room temperature, was stable for more than 4 hours.

### Example 29a

### Radiolabelling of Products (31), (34) (35), (36), (37) and (38) with Copper-64

2 mg of bis-dithiocarbamate (products 31, 34, 35, 36, 37 or 38) in 0.5 ml of ethanol are added to 1 ml of 0.1 M pH 5.5 ammonium acetate buffer containing 2 mCi of ⁶⁴Cu-acetate. The mixture is left to act for one hour. The reaction is analysed by HPLC (reverse-phase, methanol-water).

The labelling yield is greater than 95% for each of the products.

### Example 30

### Radiolabelling of Products (32), (44), (46) and (47) with TcN Leads to Products (48), (49), (50), (51) and (52)

The process is performed in the same manner as in Example 29, but with products (32), (44), (45), (46) and (47). Products (48), (49), (50), (51) and (52) are obtained.

### Example 30a

### Radiolabelling of Products (32), (44) (46) and (47) with Copper-64 Leads to Products (48), (49), (50), (51) and (52)

The process is performed in the same manner as in Example 29a, but with products (32), (44), (45), (46) and (47).

The labelling yield is greater than 95% for each of the products.

H) Examples of the Use of the Compound of the Present Invention

### Example 31

### Bioavailability of Lys-Lys Bis-Dithiocarbamate in Rats

The molecule was radiolabelled as described in Example (29). The radiolabelled compound is then isolated by HPLC, evaporated and taken up in 0.9% saline medium. The bioavailability in rats gives the following results (see Table 1):

time 30 minutes and 60 minutes: 3 animals per point,

time 3 hours: 2 animals per point,

time 24 hours: 1 animal per point.

The results are expressed as a percentage of dose injected per organ.

The rats' urine was collected 1 hour 30 minutes after injection and analysed by HPLC. The result of this analysis is 87% of unchanged complex.

TABLE 1

| | Average | Average | Average | Average |
|---|---|---|---|---|
| Organs | 30 min | 1 h | 3 h | 24 h |
| blood | 0.67 | 0.34 | 0.20 | 0.11 |
| liver | 14.38 | 9.08 | 3.84 | 1.33 |
| kidneys | 4.55 | 4.18 | 3.87 | 2.65 |
| adrenals | 0.02 | 0.01 | 0.01 | 0.00 |
| spleen | 0.13 | 0.09 | 0.05 | 0.04 |
| lungs | 0.43 | 0.28 | 0.15 | 0.08 |
| heart | 0.21 | 0.10 | 0.06 | 0.03 |
| bladder | 0.47 | 0.32 | 0.05 | 0.01 |
| urine | 14.39 | 14.70 | 0.44 | 0.05 |
| stomach | 8.24 | 3.36 | 2.64 | 0.08 |
| intestine | 20.55 | 32.92 | 51.4 | 0.49 |
| caecum | 0.31 | 0.18 | 0.11 | 0.66 |
| colon | 0.46 | 0.23 | 0.19 | 0.87 |
| brain | 0.03 | 0.01 | 0.01 | 0.01 |

### Example 32

### Biological Results of Compounds (48) and Products (50), (51) and (52)

The model chosen is the rat. Depending on the compounds, we performed one or two sacrifice times (30 minutes or 1 hour).

At the times chosen, the brains were removed and the areas of interest were isolated and counted. From these results, we deduced the following:

the crossing of the blood-brain barrier by the compound under consideration,

a striatum/cerebellum ratio.

Table II below collates the results of this experiment.

TABLE II
TcN tropane-bis-dithiocarbamate approach

| Prod- | | Biological results | | |
|---|---|---|---|---|
| uct | Precursor | Biodistribution | S/C | Kapp |
| 50 | 2β-[N^{ε},N^{ε}-bis(dithio- | 30′: cerebellum: 0.168% | 1.00 | |
| | carbamate)-N-(5-amino- | striatum: 0.167% | | |
| | hexanoyl)lysinyloxy- | crossing of blood-brain | | |
| | methyl]-3β-(4′- | barrier: 0.483% | | |
| | tolyl)tropane | 1 h: cerebellum: 0.077% | 0.97 | |
| | n = 4 | striatum: 0.075% | | |
| | m = 3 | crossing of blood-brain | | |
| | | barrier: 0.217% | | |
| 51 | 2β-[N^{δ},N^{ε}-bis(dithio- | 30′: cerebellum: 0.113% | 1.11 | |
| | carbamate)-N-(5-amino- | striatum: 0.126% | | |
| | hexanoyl)ornithyloxy- | crossing of blood-brain | | |
| | methyl]-3β-(4′- | barrier: 0.344% | | |
| | tolyl)tropane | 1 h: cerebellum: 0.069% | 1.03 | |
| | n = 3 | striatum: 0.071% | | |
| | m = 4 | crossing of blood-brain | | |
| | | barrier: 0.200% | | |
| 52 | 2β-[N^{ε},N^{δ}-bis(dithio- | 30′: cerebellum: 0.114% | 1.17 | |
| | carbamate)-N-(5-amino- | striatum: 0.133% | | |
| | pentanoyl)lysinyloxy- | crossing of blood-brain | | |
| | methyl]-3β-(4′- | barrier: 0.358% | | |
| | tolyl)tropane | 1 h: cerebellum: 0.065% | 0.98 | |
| | n = 4 | striatum: 0.064% | | |
| | m = 4 | crossing of blood-brain | | |
| | | barrier: 0.182% | | |
| 48 | 2β-[N^{ε},N^{β}-bis(dithio- | 30′: cerebellum: 0.115% | 1.09 | |
| | carbamate)(α′,δ′-di- | striatum: 0.126% | | |
| | aminobutyl)-D-lysine]- | crossing of blood-brain | | |
| | 3β-(4′-tolyl)tropane | barrier: 0.340% | | |
| | n = 4 | 1 h: cerebellum: 0.050% | 1.00 | |
| | m = 3 | striatum: 0.050% | | |
| | | crossing of blood-brain | | |
| | | barrier: 0.143% | | |

m and n are data in Table II referring to formula F.

### Example 33

### Synthesis of the Bis-Dithiocarbamate of Adrocorticotropic Hormone Fragment 1-16 (Product (53))

Ser-Tyr-Ser-Met-Glu-His-Phe-Arg-Trp-Gly-Lys-Pro-
1 5 10
Val-Gly-Lys-Lys
15

This hormone fragment of 16 amino acids contains two lysines in positions 15 and 16. We thus prepared the bis-dithiocarbamates from this fragment and according to the process of the present invention and watched how the radiolabelling took place.

0.5 mg of the adrocorticotropic hormone fragment 1-16 is dissolved in 5 ml of injection-grade water. 2 ml of piperidine are added. The mixture is left stirring for 1 hour 30 minutes. The solution is evaporated under vacuum. The residue is taken up in 5 ml of injection-grade water and 3 ml of carbon sulphide are added. The mixture is left stirring for 2 hours. The mixture is then evaporated to dryness. Product (53) consisting of the above hormone fragment comprising the bis-dithiocarbamate according to the present invention on residues 15-16 is thus obtained in freeze-dried form.

### Example 34

### Radiolabelling of Product (53) with Technetium

A flask containing a lyophilizate of product (53) is taken up in 1 ml of injection-grade water. 0.5 ml of TcO₄⁻ (20 mCi) is added to this solution. After 30 minutes, 0.2 mg of bis-dithiocarbamate (product 53) in a 0.5 M pH 7.4 phosphate buffer and optionally ethanol to dissolve the mixture are added. The manipulation is left to react for 1 hour.

PRC analysis is performed by HPLC. The labelling yield for compound 54 is greater than 95%.

### Example 35

### Reaction of Carbon Sulphide with a Monoclonal Antibody (ACM) (Product 55)

5 mg of antibody (anti-ACE) are dissolved in 3 ml of injection-grade water. 400 μl of carbon sulphide are added. The mixture is left stirring at 4° C. for 4 hours. After this time, the CS₂ is removed under vacuum at room temperature (the volume is brought to 3 ml). Product (55) is stored in solution at −18° C.

### Example 36

### Radiolabelling of the Modified Antibody (Product 56) with Technetium

Synthesis of the TCN intermediate

100 μg of tin chloride, 5 mg of SDH (succinyl dihydrazide) and 5 mg of PDTA (1,2-propanediamino-N,N,N′,N′-tetraacetic acid) were freeze-dried in a labelling flask. 3 ml of TcO₄⁻ (60 mCi) are added to this lyophilizate. This reagent is left to act for 15 minutes.

1.5 mg of antibody (1 ml) and 1.5 ml of TcN (30 mCi) are placed in a labelling flask. The mixture is left at room temperature for 1 hour. The labelling yield is checked by paper chromatography. Labelling yield for compound (56) is 93%.

### Example 37

### Formulation of a Diagnostic Kit

Flask 1: 100 μg of tin chloride, 5 mg of SDH (succinyl dihydrazide) and 5 mg of PDTA (1,2-propane-diamino-N,N,N′,N′-tetraacetic acid) were freeze-dried.

Flask 2: 2 mg of bis-dithiocarbamate (more specifically chelation complex according to the invention, containing two dithiocarbamate functions) are packaged in 1 ml of injection-grade water.

Preparation: 3 ml of TcO₄ (60 mCi) are added to flask 1. This reagent is left to act for 15 minutes and 1 ml of this solution is added to flask 2. This solution is left to act for one hour. The radiolabelling is ready for injection. Labelling yield≧95%.

The reaction is analysed by HPLC (reverse-phase, methanol-water).

1. Compound for chelating a metal or a metal complex, characterized in that it consists of a bis-dithiocarbamate structure (F) having the following formula: 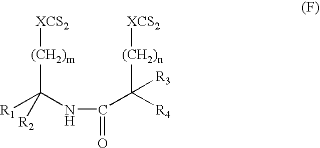
in which n and m are integers such that 5≦m+n≦10,
X is chosen independently from S and NH,
R₁, R₂, R₃ and R₄ are chosen independently from H and an organic function chosen from —COOR₅, NR₅R₆ and —CH₂OR₅ in which R₅ and R₆, when it is present, are chosen independently from a hydrogen; an amino acid; a peptide; a protein; an organic function; a group chosen from alkoxycarbonyl or aryloxycarbonyl (—COOR⁷), carboxyl (—COOH)₁ acyloxy (—O₂R⁷), carbamoyl (—CONR⁷), cyano (—CN), alkylcarbonyl, alkylarylcarbonyl, arylcarbonyl, arylalkylcarbonyl, hydroxyl (—OH), amino (NR⁷), halogen, allyl, alkoxy (—OR⁷), S-alkyl and S-aryl, R⁷ representing a C1 to C₁₀ alkyl or aryl group; an organic molecule chosen from (i) an optionally substituted alkyl, acyl, aryl or alkyne group, (ii) a saturated or unsaturated, optionally substituted or aromatic carbon-based ring or (iii) a saturated or unsaturated, optionally substituted or aromatic heterocycle, these groups and rings (i), (ii) and (iii) possibly being substituted with substituted phenyl groups, substituted aromatic groups or alkoxycarbonyl or aryloxycarbonyl (—COOR⁸); carboxyl (—COOH), acyloxy (—O₂R⁸), carbamoyl (—CONR⁸), alkylcarbonyl, alkylarylcarbonyl, arylcarbonyl, arylalkylcarbonyl, hydroxyl (—OH), amino (NR⁸), halogen, allyl, alkoxy (—OR⁸), S-alkyl or S-aryl groups, R⁸ representing a C₁ to C₁₀alkyl or aryl group; a monoclonal antibody; a hormone; and a pharmaceutically acceptable vector.

2. Chelation compound according to claim 1, in which the bis-dithiocarbamate structure consists of a structure whose formula is chosen from formulae (I), (II), (III) and (IV) below: 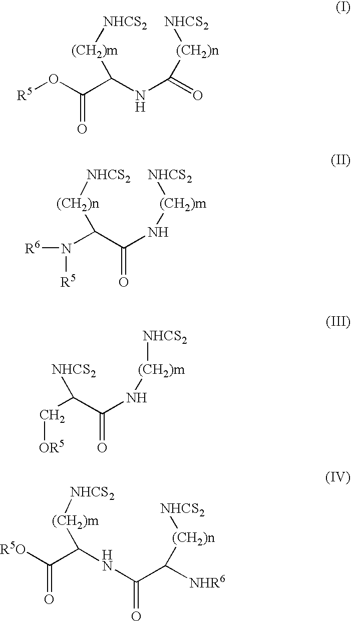
in which R⁵, R⁶, n and m, when they are present, are as defined in claim 1.

3. Compound according to claim 1 or 2, in which m and n are independently 3, 4 or 5.

4. Compound according to claim 2 or 3, in which R⁵═H.

5. Compound according to claim 2 or 3, in which R⁵═CH₃.

6. Compound according to claim 2 or 3, in which R⁵ is a tropane derivative.

7. Compound according to claim 2 or 3, in which R⁵ has the following formula:

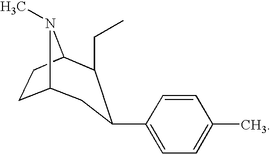

8. Compound according to claim 1, consisting of a structure of formula (F) in which R¹, R³ and R⁴═H and R² is NR⁵R⁶, in which R⁵═H and R⁶ is chosen from:

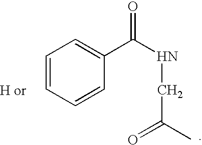

9. Chelation product consisting of a chelation compound according to any one of claims 1 to 8 and of a metal or a metal complex.

10. Chelation product according to claim 9, in which the metal is copper or an isotope thereof.

11. Chelation product according to claim 9, in which the metal is chosen from a transition metal.

12. Chelation product according to claim 9, in which the metal complex is TcN or ReN.

13. Use of a chelation compound according to any one of claims 1 to 8, for manufacturing a medicinal product or a diagnostic product.

14. Use of a chelation compound according to any one of claims 1 to 8, for manufacturing a radiopharmaceutical for therapy or for diagnosis.

15. Use of a chelation compound according to any one of claims 1 to 8, for manufacturing a radiopharmaceutical for visualizing the uptake of dopamine or serotonin.

16. Use of a chelation product according to any one of claims 9 to 12, for manufacturing a medicinal product or a diagnostic product.

17. Use of a chelation product according to any one of claims 9 to 12, for manufacturing a radiopharmaceutical for therapy or diagnosis.

18. Use of a chelation product according to any one of claims 9 to 12, for manufacturing a radiopharmaceutical for visualizing the uptake of dopamine or serotonin.

19. Process for manufacturing a bis-dithiocarbamate structure as defined in claim 1, comprising, successively:
a step of protecting the XH functions of the compounds of formulae (V) and (VI) below: 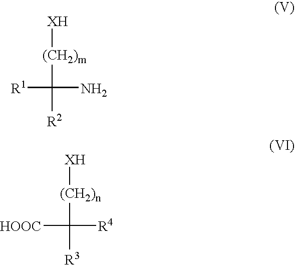
in which R¹, R², R³, R⁴, X, m and n are as defined in claim 1,
a step of activating the —COOH function of compound (VI),
a step of linking the compound of formula (V) and compound (VI) via the activated carboxyl function of compound (VI),
a step of deprotecting the XH functions, and
a step of reacting the deprotected XH functions with CS₂ to form a bis-dithiocarbamate structure according to claim 1.

20. Process for manufacturing a structure according to claim 1 or 2, comprising a process according to claim 19 for manufacturing a bis-dithiocarbamate structure, and also comprising a step of attaching a radical R⁵ and optionally R⁶ to this bis-dithiocarbamate structure or to an intermediate product in its manufacture to obtain a chelation compound according to claim 1 or 2.

21. Process for manufacturing a structure as defined in claim 1 or 2, comprising:
a step of reacting two ε-NH₂ functions of two contiguous amino acids of a precursor molecule of the structure according to claim 1 or 2 with CS₂ so as to form a structure according to claim 1 or 2,
the precursor molecule constituting the radical R⁵ and optionally the radical R⁶.

22. Process for preparing a chelation product defined in claim 9, comprising the manufacture of a bis-dithiocarbamate structure defined in claim 1 or 2 according to a manufacturing process defined in claim 20 or 21, and a reaction for complexing a metal or a metal complex via the said bis-dithiocarbamate structure manufactured.

23. Process according to claim 22, in which the metal is a transition metal.

24. Process according to claim 22, in which the metal is copper.

25. Process according to claim 22, in which the metal complex is TcN or ReN.

26. Diagnostic kit comprising a chelating compound according to any one of claims 1 to 8.
